(19) United States
(12) Patent Application Publication
(10) Pub. No.: US 2024/0127920 A1
(43) Pub. Date: Apr. 18, 2024

(54) MEDICAL EXAMINATION AND PRESCRIPTION APPARATUS FOR HERBAL MEDICINE PRESCRIPTION AND METHOD THEREFOR

(71) Applicant: Euy Joon Roh, Gyeonggi-do (KR)

(72) Inventor: Euy Joon Roh, Gyeonggi-do (KR)

(21) Appl. No.: 18/379,143

(22) Filed: Oct. 11, 2023

(30) Foreign Application Priority Data
Oct. 12, 2022 (KR) 10-2022-0130286

(51) Int. Cl. G16H 20/10; G16H 10/20

(52) U.S. Cl. CPC G16H 20/10; G16H 10/20

(57) ABSTRACT

The present invention is related to a medical examination and prescription apparatus for herbal medicine prescription, and a method therefor, wherein first of all, a herbal medicine mapped with a medical examination is extracted through the medical examination; a herbal medicine formulation related to the extracted herbal medicine is extracted to generate a first prescription candidate group, and secondly, five prescription evidences mapped with a medical examination are extracted through the medical examination; and a prescription pool list is provided by differentially applying a second prescription candidate group obtained by correlation mapping the extracted prescription evidence to the first prescription candidate group according to a grade and a rank.

### CROSS-REFERENCE TO RELATED APPLICATION

This application claims priority of Korean Patent Application No. 10-2022-0130286, filed on Oct. 12, 2022, in the KIPO (Korean Intellectual Property Office), the disclosure of which is incorporated herein entirely by reference.

### BACKGROUND OF THE INVENTION

### Field of the Invention

The present invention relates to a medical examination and prescription apparatus for herbal medicine prescription and a method therefor, and more particular, relates to a medical examination and prescription apparatus for herbal medicine prescription and a method therefor, wherein first of all, a herbal medicine mapped with a medical examination is extracted through the medical examination, a herbal medicine formulation related to the extracted herbal medicine is extracted to generate a first prescription candidate group, and secondly, five prescription evidences mapped with a medical examination are extracted through the medical examination, and a prescription pool list is provided by differentially applying a second prescription candidate group obtained by correlation mapping the extracted prescription evidence to the first prescription candidate group according to a grade and a rank.

### Description of the Related Art

The invention disclosed in prior patent document KR 10-2044079 discloses a method and apparatus for searching oriental medicine prescription. That is, the apparatus for searching for oriental medicine prescription disclosed in prior patent documents provides a user interface for searching for oriental medicine prescription, and receives first search information for medicinal materials or prescription to be included in the search results through the user interface, receive the second search information on medicine or prescription to be excluded from the search results through the user interface, receive a search condition corresponding to at least one of the number of medicine, dosage form, and a source through the user interface, and search a database linking the prescription provisions arranging the contents recorded in the source according to prescription, and linking the prescription and prescribed medicine substances. As a result, at least one candidate prescription including the medicine or prescription of the first search information and excluding the medicine or prescription of the second search information is extracted, and at least one search result prescription which satisfies the search condition is selected from the extracted candidate prescription may be provided.

The candidate prescription disclosed in the prior patent document are extracted when a user inputs a drug to be artificially included, the number of drugs, a formulation, and a source into a search condition, and the input search condition is matched with the candidate prescription. The candidate prescription disclosed in these prior patent documents merely provide a user interface and database for searching oriental medicine prescription regardless of the patient's symptoms or conditions, and a new method for automatically generating candidate prescription according to the patient's symptoms is required.

### SUMMARY OF THE INVENTION

Therefore, the present invention was created to solve the above-described problems, and an object of the present invention is to provide an invention that may provide standardized and quantified herbal medicine formulation to patients or oriental doctors in the same business through medical questionnaires.

However, the objects of the present invention are not limited to the above-mentioned objects, and other undisclosed objectives can be clearly understood by those skilled in the art from the description below.

The object of the present invention described above is achieved by providing a medical examination and prescription apparatus for herbal medicine prescription, comprising: a medical examination unit that conducts a medical examination in question-and-answer with a patient step by step according to a pre-determined order and receives an answer to question from the patient according to symptoms of a disease; a first prescription candidate group deriving unit for sequentially extracting herbal medicines related to the medical examination according to the medical examination and deriving a first prescription candidate group of herbal medicine formulation corresponding to the extracted herbal medicines; a second prescription candidate group deriving unit for mapping prescription evidence to the first prescription candidate group of herbal medicine formulation through the sequential medical examination and deriving a second prescription candidate group of herbal medicine formulation by differentiating the first prescription candidate group of herbal medicine formulation according to a grade and a rank; and a prescription providing unit for providing an information corresponding to the herbal medicine formulation prescribed by differentially listing according to the grade and the rank.

In addition, the first prescription candidate group extraction unit may include a herbal medicine extraction unit which sequentially extracts a herbal medicine to be included in the herbal medicine formulation and herbal medicines to be excluded from the herbal medicine formulation from a herbal medicine database unit at each stage of the medical examination during the sequential medical examination. It also comprises a first prescription candidate group extraction unit that sequentially extracts the first prescription candidate group of herbal medicine formulations containing the herbal medicine extracted from the herbal medicine extraction unit from a herbal medicine formulation database unit.

In addition, the second prescription candidate group extraction unit may include a prescription evidence mapping unit which sequentially maps five types of prescription evidence to the first prescription candidate group of herbal medicine formulation extracted from the first prescription candidate group extraction unit through the sequential medical examination based on an input from the herbal medicine formulation database unit; and a second prescription candidate group extraction unit which extracts the second prescription candidate group of herbal medicine formulation by differentiating the first prescription candidate group of herbal medicine formulation mapped by the prescription evidence mapping unit based on a grade and a rank according to a degree of mapping of the five types of prescription evidence.

In addition, the second prescription candidate group extraction unit may further include a prescription evidence grade determination unit which provides a grade and a rank criteria. This allows for the determination of the grade and rank of the mapped first prescription candidate group of herbal medicine formulation based on the frequency of clinical stochastic symptoms of essential symptoms, frequent symptoms, infrequent symptoms, tendencies, and avoidance symptoms.

In addition, the essential symptoms, the frequent symptoms and the avoidance symptoms may serve as criteria for determining a grade, while the infrequent symptoms and the tendencies may serve as criteria for determining a rank. A prescription evidence grade determination unit may primarily determine the rank based on the grade. In cases where the grade is the same, it may determine the rank based on the infrequent symptoms and the tendencies.

In addition, the herbal medicine formulation database unit may include herbal medicine formulation data related to a herbal medicine prescription and data for five types of prescription evidence, each of which has a correlation with each herbal medicine formulation.

In addition, the medical examination and prescription apparatus may further include a visiting patient response evaluation unit that receives responses of a questionnaire related to a prescription from an existing patient who has received the prescription. It classifies and evaluates the input responses of the questionnaire according to a type of symptom. It also includes a prescription error learning unit which learns prescription errors based on medical examination, herbal medicine extraction, prescription evidence mapping, and prescription evidence grade according to the classification and evaluation of the questionnaire responses from the visiting patient response evaluation unit.

Furthermore, the medical examination and prescription apparatus may further include a medical examination correction unit which corrects the existing medical examination based on medical examination error data provided by the prescription error learning unit and provides it to the medical examination unit; a herbal medicine extraction error correction unit which corrects the herbal medicine extracted in correlation with the medical examination based on herbal medicine extraction error data provided by the prescription error learning unit and provides it to the herbal medicine extraction unit; a prescription evidence mapping error correction unit which adds new learning prescription evidence to the five types of prescription evidence based on prescription evidence mapping error data provided by the prescription error learning unit and provides it to the prescription evidence mapping unit; and a prescription evidence grade error correction unit which modifies the grade and rank criteria of the five types of prescription evidence based on prescription evidence grading error data provided by the prescription error learning unit and provides them, or adds a grade and rank criteria of the new learning prescription evidence added by the prescription evidence mapping error correction unit and provides them to the prescription evidence grade determination unit.

On the other hand, the objects of the present invention may be achieved by providing a medical examination and prescription method for herbal medicine prescription. The method may include the following steps: conducting a step-by-step medical examination in question-and-answer format with a patient through a medical examination unit, following a pre-determined order, receiving the patient's answers based on disease symptoms; a step for sequentially extracting a herbal medicine to be included in a herbal medicine formulation and a herbal medicine to be excluded from the herbal medicine formulation from a herbal medicine database unit in each stage of the medical examination according to the sequential medical examination by using a herbal medicine extraction unit; a step for sequentially extracting a first prescription candidate group of herbal medicine formulation containing the herbal medicine extracted from the herbal medicine extraction unit from a herbal medicine formulation database unit by using a first prescription candidate group extraction unit; a step in which a prescription evidence mapping unit sequentially maps five types of prescription evidence to the first prescription candidate group of herbal medicine formulation extracted from the first prescription candidate group extraction unit through the sequential medical examination based on an input of the herbal medicine formulation database unit; a step in which a second prescription candidate group extraction unit differentiates the first prescription candidate group of herbal medicine formulation mapped by the prescription evidence mapping unit by a grade and a rank according to a degree of mapping of the five types of prescription evidence, and extracts a second prescription candidate group of herbal medicine formulation; a step in which a prescription providing unit provides an information corresponding to the herbal medicine formulation prescribed by differentially listing according the grade and the rank; a step in which a visiting patient response evaluation unit receives responses of a questionnaire related to a prescription from an existing patient who has received the prescription, and classifies and evaluates the input responses of the questionnaire according to a type of symptom; a step in which a prescription error learning unit learns a prescription error, corrects and complements it based on medical examination, herbal medicine extraction, prescription evidence mapping, and prescription evidence grade according to the classification and evaluation of the questionnaire responses of the visiting patient response evaluation unit; a step in which a medical examination correction unit corrects the existing medical examination based on medical examination error data provided by the prescription error learning unit, and provides it to the medical examination unit; a step in which a herbal medicine extraction error correction unit corrects the herbal medicine extracted in correlation with the medical examination based on herbal medicine extraction error data provided by the prescription error learning unit, and provides it to the herbal medicine extraction unit; a step in which a prescription evidence mapping error correction unit adds new learning prescription evidence to the five types of prescription evidence based on prescription evidence mapping error data provided by the prescription error learning unit, and provides it to the prescription evidence mapping unit; a step in which a prescription evidence grade error correction unit corrects a grade and rank criteria for the five types of prescription evidence based on prescription evidence grading error data provided by the prescription error learning unit and provides them, or adds a grade and rank criteria of the new learning prescription evidence added by the prescription evidence mapping error correction unit, and provides them to a prescription evidence grade determination unit; and a step for correcting and deriving the first prescription candidate group and the second prescription candidate group of herbal medicine formulation step by step.

According to the present invention as described above, there is an effect that standardized and quantified herbal medicine formulations may be provided to patients or oriental doctors in the same business through medical examination (question-and-answer).

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a diagram schematically showing a medical examination and prescription apparatus for herbal medicine prescription according to an embodiment of the present invention.

FIG. 2 is a diagram illustrating a case that the herbal medicine correlated with the medical examination is derived through the medical examination according to an embodiment of the present invention.

FIG. 3 is a diagram illustrating a case that five prescription evidences correlated with the medical examination are derived through the medical examination according to an embodiment of the present invention, and the derived prescription evidence is correlation-mapped to the first prescription candidate group.

FIG. 4 is a diagram illustrating correction and complementation of prescription errors based on medical examination, herbal medicine extraction, prescription evidence mapping, and a prescription evidence grade through learning according to an embodiment of the present invention.

FIG. 5 is a view sequentially illustrating a medical examination and prescription method for herbal medicine prescription according to an embodiment of the present invention.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

A medical examination and prescription apparatus for herbal medicine prescription, according to an embodiment of the present invention, is, for example, an apparatus that allows a patient to visit a hospital or remotely access a hospital through an application running on a computer, laptop, tablet PC, smart phone terminal, etc., to prescribe herbal medicine through medical examination. For example, a web platform capable of conducting medical examination (question-and-answer) and prescription may be built in a computer, and an application capable of performing the same function as the web platform may be installed in a smart phone terminal. Hereinafter, a medical examination and prescription apparatus for herbal medicine prescription, according to an embodiment of the present invention, will be described in detail with reference to the accompanying drawings.

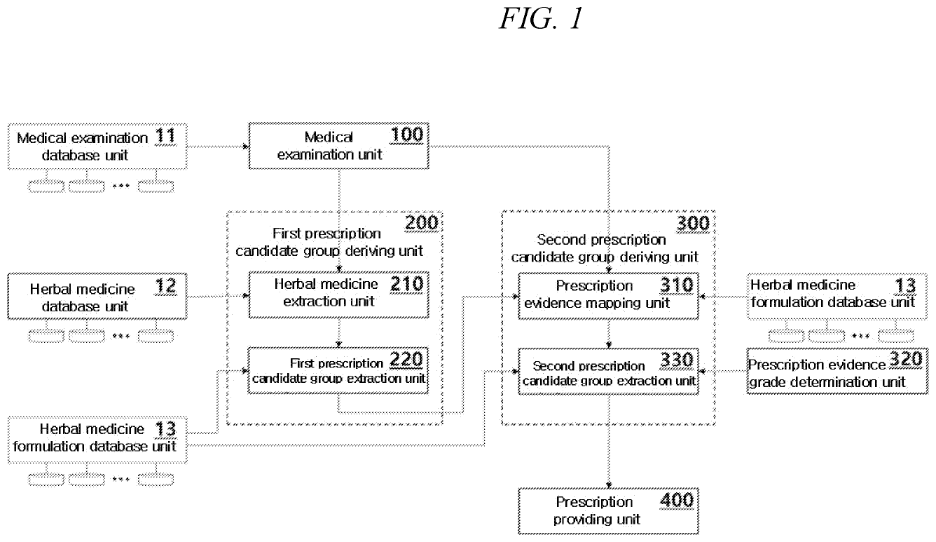

As shown in FIG. 1, a medical examination unit 100, according to an embodiment of the present invention, uses approximately 180 questions stored in a medical examination database unit 11 to conduct a medical examination in question-and-answer with a patient step by step according to a pre-determined order, and receives an answer to question from the patient according to symptoms of a disease. The order in which questions are posed to the patient may be configured to ask questions sequentially, covering mental questions, questions related to input into or output from the human body, and temperature (whether the patient feels cold or hot and the temperature of hands, feet, and lower abdomen). The medical examination data may undergo standardization and quantification and may be adjusted based on patient feedback, as described below.

A first prescription candidate group deriving unit 200, according to an embodiment of the present invention, derives herbal medicine data by sequentially extracting a herbal medicine (clue drug, Guiding Herb) related to medical examination data and it also derives a first prescription candidate group of herbal medicine formulation data related to the derived herbal medicine data, based on the medical examination data transmitted from the medical examination unit 100 (here, the medical examination data is the patient's answer to the questions). The herbal medicine data is derived by using a herbal medicine database unit 12, and the first prescription candidate group data of the herbal medicine formulation is derived by using a herbal medicine formulation database unit 13 (or a herbal medicine prescription database unit).

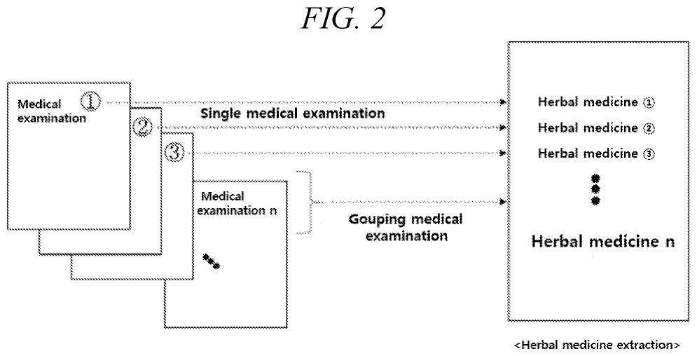

In the herbal medicine database unit 12, the herbal medicine which is mutually matching with the medical examination data are defined in a matching manner. As an example, the herbal medicine related to medical question-and-answer 1 are defined, the herbal medicine related to medical question-and-answer 2 are defined, and the herbal medicine related to medical question-and-answers 5 and 6 (this is called a grouped medical question-and-answer) may be defined. That is, the herbal medicine to be extracted may be extracted from a single medical question-and-answer, or may be extracted from grouped medical question-and-answers which are continuous and related to each other. Therefore, referring to FIG. 2, if the medical examination is performed sequentially and step by step by using a single medical question-and-answer (questionnaire) and a grouped medical question-and-answer (questionnaire), necessary herbal medicine data may be derived. At this time, the herbal medicine included in the derived herbal medicine data are matched with the medical examination data so that necessary herbal medicine may be included through a single medical question-and-answer and a grouped medical question-and-answer, and the herbal medicine which must be left out are matched with the medical examination data so that they may be excluded from the derived herbal medicine data.

The first prescription candidate group deriving unit 200 includes a herbal medicine extraction unit 210 and a first prescription candidate group extraction unit 220. The herbal medicine extraction unit 210 sequentially extracts the herbal medicine to be included in the herbal medicine formulation and the herbal medicine to be excluded from the herbal medicine database unit 12 by using the medical examination data obtained according to the sequential medical examination of the medical examination unit 100 to create herbal medicine data. The first prescription candidate group extraction unit 220 extracts and generates herbal medical formulation data of the first prescription candidate group by matching and comparing the herbal medicine data extracted from the herbal medicine extraction unit 210 and the herbal medicine formulation that is mutually matched by using the herbal medicine formulation database unit 131.

The herbal medicine formulation database unit 13 includes herbal medicine formulation data related to a herbal medicine prescription, the herbal medicine formulation data which is mutually matched with the herbal medicine formulation data, and five types of prescription evidence data correlated with each of the herbal medicine formulation. Herbal medicine prescription standard data capable of presenting a standard for herbal medicine prescription is stored in the herbal medicine formulation database unit 13. As an example, in the data table of ‘A herbal medicine formulation’, there are ‘a herbal medicine’ data table of ‘a={a1, a2, a3}’ matched with ‘A herbal medicine formulation’, and ‘five types of prescription evidence’ data table of ‘t={t1, t2, t3, t4, t5}’. Therefore, it is possible to derive a plurality of herbal medicine formulation related to the extracted herbal medicine by comparing the various types of herbal medicine data extracted from the herbal medicine extraction unit 210 with the herbal medicine database unit 12.

A second prescription candidate group deriving unit 300 according to an embodiment of the present invention extracts creates herbal medicine formulation data in which prescription evidence is mapped by mapping five types of prescription evidence to the herbal medicine data of the first prescription candidate group extracted from the first prescription candidate group extraction unit 220 through the sequential medical examination of the medical examination unit 100 by using the herbal medicine formulation database unit 13, derives a herbal medicine formulation data of a second prescription candidate group by differentially applying the herbal medicine formulation data to which the prescription evidence is mapped according to a grade and a rank, and finally creates a prescription list.

To this end, the second prescription candidate group deriving unit 300 includes a prescription evidence mapping unit 310, an prescription evidence grade determination unit 320, and a second prescription candidate group extraction unit 330.

The prescription evidence mapping unit 310 sequentially correlation-maps five types of prescription evidence (e.g., essential symptom, frequent symptom, infrequent symptom, tendency, and avoidance symptom) to the herbal medicine formulation data of the first prescription candidate group extracted from the first prescription candidate group extraction unit 220 through sequential medical examination of the medical examination unit 100 by using the herbal medicine formulation database 13. At this time, the essential symptom is related to a medical examination that must be present when prescribing a prescription, the frequent symptom is related to a medical examination that frequently appears when prescribing a prescription, the infrequent symptom is relate to a medical examination that sometimes (rarely) appears when prescribing a prescription, the tendency is related to a phenomenon which may appear when prescribing a prescription, and the avoidance symptom is related to a medical examination that should not appear when prescribing a prescription.

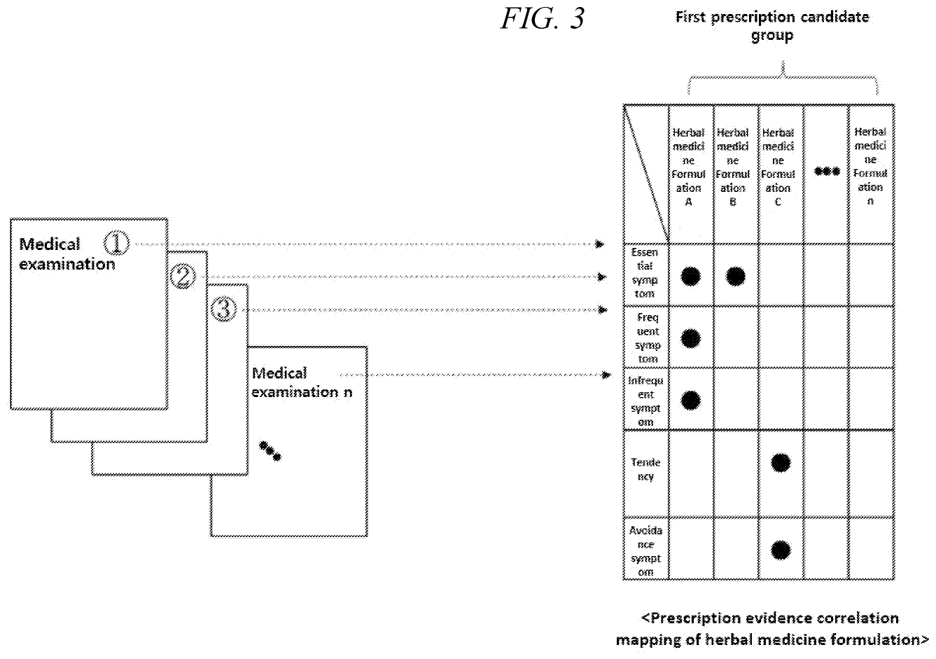

That is, if an example is explained with referring to FIG. 3, the prescription evidence derived through the medical examination is mapped to the herbal medicine formulation data table of the first prescription candidate group extracted by the first prescription candidate group extraction unit 220, so that it may have correlations. As an example, ‘A herbal medicine formulation’ is basically a herbal medicine formulation for treating symptoms of essential symptom, frequent symptom, infrequent symptom, and avoidance symptom, and if correlation mapping of prescription evidences derived through medical examination is performed, essential symptom, frequent symptom, infrequent symptom may be mapped. In addition, the ‘B herbal medicine formulation’ is basically a herbal medicine formulation for treating symptoms of essential symptom, frequent symptom, and avoidance symptom, and ‘essential symptom’ may be mapped by correlation mapping of the prescription evidence derived through the medical examination. In addition, ‘C herbal medicine formulation’ is basically a herbal medicine formulation for treating frequent symptom, avoidance symptom, and tendency, and ‘tendency and avoidance symptom’ may be mapped by correlation mapping of prescription evidence derived through medical examination.

In this way, in ‘A herbal medicine formulation’, ‘essential symptoms, frequent symptoms, and infrequent symptoms’ are correlated through the correlation mapping process. Within ‘B herbal medicine formulation’, ‘essential symptom’ is mapped, and in ‘C herbal medicine formulation’, ‘tendency and avoidance symptom’ are finally mapped. According to the correlation mapping of a prescription evidence of the first prescription candidate group, the herbal medicine formulation data of the second prescription candidate group according to a grade and a rank may be derived as described below, and a prescription list may be provided according to a grade and a rank.

On the other hand, the medical examination unit 100 may include two types of medical examination. That is, a first medical examination (questionnaire) defined to derive herbal medicine data and a second medical examination (questionnaire) defined to derive prescription evidence may be included. In addition, any one medical examination or grouped medical examination may include a medical examination capable of deriving both herbal medicine data and prescription evidence. The first medical examination and the second medical examination are stored in the medical examination database unit 11. The order of the first medical examination and the second medical examination may be variously changed based on the patient's type or symptom or the patient's past history.

The prescription evidence grade determination unit 320 provide a grade and a rank criterion to the second prescription candidate group extraction unit 330 to determine a grade and a rank of the herbal medicine formulation data of the first prescription candidate group obtained by correlation-mapping the prescription evidence and the medical examination constructed on the basis of the frequency of clinical stochastic symptom appearance of ‘essential symptom, frequent symptom, infrequent symptom, tendency, and avoidance symptom. Among the five types of prescription evidences, ‘essential symptom, frequent symptom and avoidance symptom’ are the criterion for determining the grade, ‘infrequent symptom and tendency’ are criteria for determining a rank, the prescription evidence grade determination unit 320 primarily determines the ranks based on grades, and in the same grade, determines the ranks based on infrequent symptom and tendency.

The second prescription candidate group extraction unit 330 differentiates the herbal medicine formulation data of the first prescription candidate group in which the medical examination and the prescription evidence are correlation-mapped to each other by the prescription evidence mapping unit 310 by indicating grades and ranks according to the degree of mapping of the correlation mapping, so that the herbal medicine formulation data of the second prescription candidate group may be derived. That is, referring to an example of FIG. 3, in the correlation-mapped herbal medicine formulation data table, ‘essential symptom, frequent symptom, and infrequent symptom’ are correlation-mapped in ‘A herbal medicine formulation’, an ‘essential symptom’ is correlation-mapped in ‘B herbal medicine formulation’, and ‘tendency and avoidance symptom’ is correlation-mapped in ‘C herbal medicine formulation’. Therefore, first of all, if a rank is designated based on ‘a grade’, ‘A herbal medicine formulation’ is derived as the highest grade, differentially followed by ‘B herbal medicine formulation’ and ‘C herbal medicine formulation’. ‘A herbal medicine formulation and B herbal medicine formulation’ may be of the same grade, but ‘A herbal medicine formulation’ has higher priority because more correlation mapping is being done in the same grade, and ‘B herbal medicine formulation’ is higher grade than ‘C herbal medicine formulation’.

A prescription providing unit 400 may provide a prescription pool list by differentially listing the herbal medicine formulation data of the differentially applied second prescription candidate group extracted by the second prescription candidate group extraction unit 330 according to a grade and a rank. That is, the prescription providing unit 400 may provide an information corresponding to the herbal medicine formulation prescribed by differentially listing according to the grade and the rank.

On the other hand, a medical examination, herbal medicine extraction, prescription evidence mapping, and prescription evidence grade criterion according to an embodiment of the present invention may be modified through a questionnaire survey of patients who have received a prescription. In the following, this will be explained.

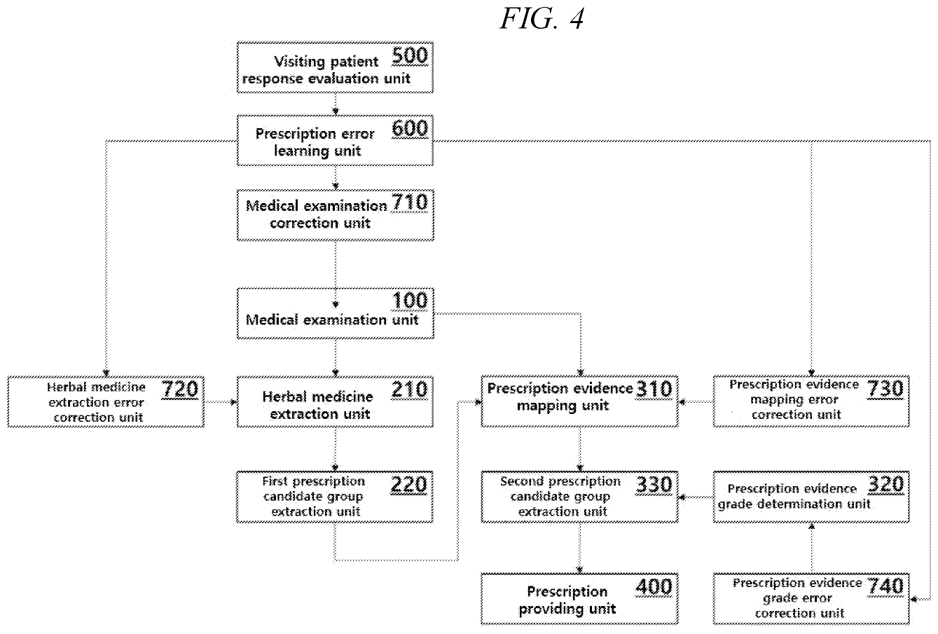

Referring to FIG. 4, a visiting patient response evaluation unit 500 according to an embodiment of the present invention receives responses of a questionnaire related to the prescription from an existing patient who has received a prescription, categorizes and classifies the input responses of the questionnaire according to the type of symptom, and evaluates them. That is, as an example, whether or not the content of responses of the questionnaire received from the patient is sufficiently reliable to receive feedback is discriminated and evaluated according to the criterion.

A prescription error learning unit 600 according to an embodiment of the present invention learning-evaluates a questionnaire determined to be reliable according to the classification and evaluation of the questionnaire responses of the visiting patient response evaluation unit 500 based on medical examination, herbal medicine extraction, prescription evidence mapping, and prescription evidence grade, and generates a prescription error correction data based on medical examination, herbal medicine extraction, prescription evidence mapping, and prescription evidence grade according to the learning evaluation.

A medical examination correction unit 710 according to an embodiment of the present invention corrects the existing medical examination based on medical examination error data of the prescription error correction data provided from the prescription error learning unit 600 and provides it to the medical examination unit 100. The medical examination unit 100 records the corrected medical examination in the questionnaire database unit 11. The corrected medical examination may be the order of the medical examination (questionnaire), or a new medical examination (questionnaire) may be added or an existing medical examination (questionnaire) may be excluded.

A herbal medicine extraction error correction unit 720 according to an embodiment of the present invention corrects the herbal medicine extracted in correlation with the medical examination based on the herbal medicine extraction error data of the prescription error correction data provided by the prescription error learning unit 600, and provides it to the herbal medicine extraction unit 210. For example, in the past, when ‘a herbal medicine and b herbal medicine’ are extracted as the herbal medicine related to the first medical examination (questionnaire), the corrected herbal medicine may be modified so that ‘a herbal medicine and c herbal medicine’ may be extracted. Herbal medicine extracted in correlation with the medical examination is corrected and recorded in the herbal medicine database unit 12.

A prescription evidence mapping error correction unit 730 according to an embodiment of the present invention adds new ‘learning prescription evidence’ to the five types of prescription evidence based on the prescription evidence mapping error data of the prescription error correction data provided from the prescription error learning unit 600, and provides it to the prescription evidence mapping unit 310. As another example, the correlations of prescription evidence extracted through a medical examination may be set differently. That is, if the correlation has been previously mapped so that ‘essential symptom and frequent symptom’ may be derived through ‘A medical examination (questionnaire)’, it may be modified to derive ‘frequent symptom’ through ‘A medical examination (questionnaire)’ after correction. The newly added ‘learning prescription evidence’ is additionally recorded in the herbal medicine formulation database unit 13.

A prescription evidence grade error correction unit 740 according to an embodiment of the present invention corrects the grade and rank criteria of the five types of prescription evidence based on the prescription evidence grading error data of the prescription error correction data provided by the prescription error learning unit 600 and provides them, or adds a grade and rank criteria of the new learning prescription evidence added by the prescription evidence mapping error correction unit 730 and provides them to the prescription evidence grade determination unit 320. The prescription evidence grade determination unit 320 provides the newly defined grade and rank to the second prescription candidate group extraction unit 330 so that the second prescription candidate group extraction unit 330 may generate the priority of the prescription according to a grade/a rank.

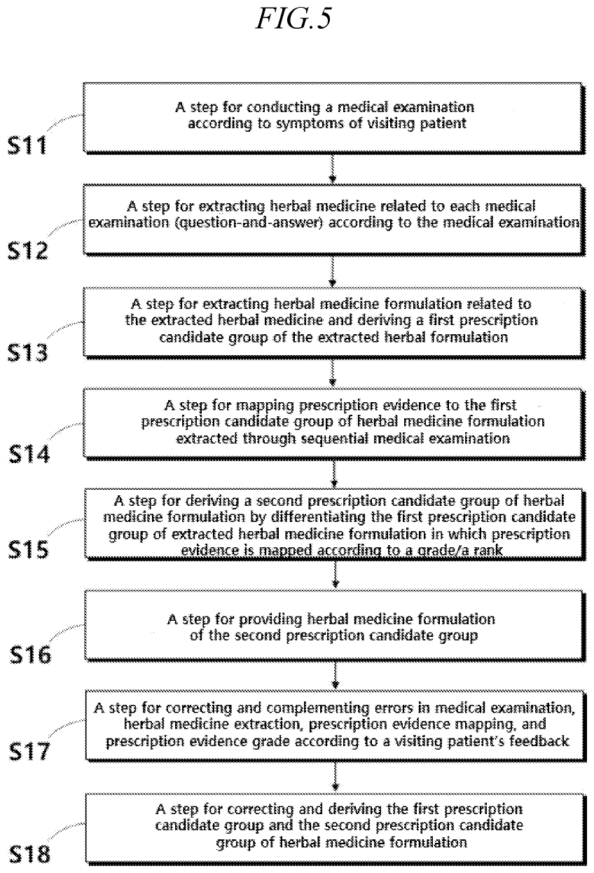

The medical examination and prescription method for herbal medicine prescription according to an embodiment of the present invention may be performed according to the following method and sequence, as shown in FIG. 5.

First of all, the medical examination unit 100 conducts a medical examination in question-and-answer with a patient step by step according to a pre-determined order, receives an answer to question from the patient according to symptoms of a disease, and generates medical examination data. At this time, the medical examination unit 100 performs a TIOM medical examination (questionnaire) to preemptively prescribe a herbal medicine prescription (a suitable prescription) which are most suitable for the patient's type of disease and physical condition pattern. The order of TIOM medical examination (questionnaire) may be changed as needed, and the number of questions may also be increased or decreased as needed. On the other hand, the TIOM medical examination (questionnaire) is classified into heat (temperature), eating (In take, for example, appetite, digestion, drinking water), excretion (output, for example, feces, urine, sweat, menstruation), life/chest symptoms (mental), the questions are provided sequentially, and the medical examination is completed when the patient answers to the questions according to his/her condition.

Next, the herbal medicine extraction unit 210 sequentially extracts a herbal medicine to be included in a herbal medicine formulation and a herbal medicine to be excluded from the herbal medicine formulation from the herbal medicine database unit 12 in each stage of the medical examination according to the sequential medical examination, and generates the herbal medicine data.

Next, the first prescription candidate group extraction unit 220 sequentially derives the herbal medicine formulation of the first prescription candidate group containing the herbal medicine extracted from the herbal medicine extraction unit 210 from the herbal medicine formulation database 13, and generates a herbal medicine formulation data of the first prescription candidate group.

Next, the prescription evidence mapping unit 310 generates a herbal medicine formulation data table of the first prescription candidate group in which the prescription evidence is correlated by sequentially mapping the five types of prescription evidence derived based on the correlation through the medical examination to the herbal medicine formulation data extracted from the first prescription candidate group extraction unit 220, based on the input of the herbal medicine formulation database unit 13.

Next, the second prescription candidate group extraction unit 330 differentially applies by a grade and a rank according to a degree of mapping of the five types of prescription evidence by using the herbal medicine formulation data of the first candidate group to which the prescription evidence is correlation-mapped by the prescription evidence mapping unit 310, and then derives and generates herbal medicine formulation data of second prescription candidate group.

Next, the prescription providing unit 400 lists prescription pool by differentially applying according to a grade and a rank, and provides the prescription pool to the patient. That is, the prescription providing unit 400 may provide an information corresponding to the herbal medicine formulation prescribed by differentially listing according the grade and the rank.

Next, the visiting patient response evaluation unit 500 receives responses of a questionnaire related to a prescription from an existing patient who received the prescription, classifies the input responses of the questionnaire according to the type of symptom, and evaluates reliability.

Next, the prescription error learning unit 600 learns the prescription error based on the medical examination, herbal medicine extraction, prescription evidence mapping, and prescription evidence grade according to the classification and reliability evaluation of the questionnaire responses of the visiting patient response evaluation unit 500 to create error correction data.

Next, the medical examination correction unit 710 corrects the existing medical examination based on the medical examination error data provided by the prescription error learning unit 600 and provides it to the medical examination unit 100. The herbal medicine extraction error correction unit 720 corrects the herbal medicine extracted in correlation with the medical examination based on the herbal medicine extraction error data provided by the prescription error learning unit 600, and provides it to the herbal medicine extraction unit 210. The prescription evidence mapping error correction unit 730 adds a new learning prescription evidence to the five types of prescription evidence based on the prescription evidence mapping error data provided by the prescription error learning unit 600, or corrects the correlation between the questionnaire and the prescription evidence, and provides it to the prescription evidence mapping unit. The prescription evidence grade error correction unit 740 corrects and provides a grade and rank criterion of the five types of prescription evidence based on prescription evidence grade error data provided by the prescription error learning unit 600, or adds a grade and rank criterion of the new learning prescription evidence added by the prescription evidence mapping error correction unit 740, and provides them to the prescription evidence grade determination unit 320.

Next, the first prescription candidate group and the second prescription candidate group are sequentially modified (corrected) and derived step by step by using the error correction data derived by the learning evaluation of the prescription error learning unit 600, and finally, a prescription pool list modified by the prescription providing unit 400 is generated.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A medical examination and prescription apparatus for herbal medicine prescription, comprising:
a medical examination unit that conducts a medical examination in question-and-answer with a patient step by step according to a pre-determined order and receives an answer to question from the patient according to symptoms of a disease;
a first prescription candidate group deriving unit for sequentially extracting herbal medicines related to the medical examination according to the medical examination and deriving a first prescription candidate group of herbal medicine formulation corresponding to the extracted herbal medicines;
a second prescription candidate group deriving unit for mapping prescription evidence to the first prescription candidate group of herbal medicine formulation through the sequential medical examination and deriving a second prescription candidate group of herbal medicine formulation by differentiating the first prescription candidate group of herbal medicine formulation according to a grade and a rank; and
a prescription providing unit for providing an information corresponding to the herbal medicine formulation prescribed by differentially listing according to the grade and the rank.

2. The medical examination and prescription apparatus for herbal medicine prescription of claim 1, wherein the first prescription candidate group deriving unit comprising:
a herbal medicine extraction unit which sequentially extracts a herbal medicine to be included in the herbal medicine formulation and a herbal medicine to be excluded from the herbal medicine formulation from a herbal medicine database unit in each stage of the medical examination according to the sequential medical examination; and
a first prescription candidate group extraction unit which sequentially extracts the first prescription candidate group of herbal medicine formulation containing the herbal medicine extracted from the herbal medicine extraction unit from a herbal medicine formulation database unit.

3. The medical examination and prescription apparatus for herbal medicine prescription of claim 2, wherein the second prescription candidate group deriving unit comprising:
a prescription evidence mapping unit which sequentially maps five types of prescription evidence to the first prescription candidate group of herbal medicine formulation extracted from the first prescription candidate group extraction unit through the sequential medical examination based on an input of the herbal medicine formulation database unit; and
a second prescription candidate group extraction unit which extracts the second prescription candidate group of herbal medicine formulation by differentiating the first prescription candidate group of herbal medicine formulation mapped by the prescription evidence mapping unit based on a grade and a rank according to a degree of mapping of the five types of prescription evidence.

4. The medical examination and prescription apparatus for herbal medicine prescription of claim 3, wherein the second prescription candidate group extraction unit further comprising a prescription evidence grade determination unit which provides a grade and a rank criteria, so that a grade and a rank of the mapped first prescription candidate group of herbal medicine formulation may be determined based on frequency of clinical stochastic symptoms of essential symptom, frequent symptom, infrequent symptom, tendency and avoidance symptom.

5. The medical examination and prescription apparatus for herbal medicine prescription of claim 4,
wherein the essential symptom, the frequent symptom, and the avoidance symptom are criteria for determining a grade, and the infrequent symptom, and the tendency are criteria for determining a rank,
wherein the prescription evidence grade determination unit primarily determines the rank based on the grade, and in the same grade, determines the rank based on the infrequent symptom and the tendency.

6. The medical examination and prescription apparatus for herbal medicine prescription of claim 3, wherein the herbal medicine formulation database unit includes a herbal medicine formulation data related to a herbal medicine prescription and five types of prescription evidence data having a correlation with each herbal medicine formulation.

7. The medical examination and prescription apparatus for herbal medicine prescription of claim 4, further comprising:
a visiting patient response evaluation unit that receives responses of a questionnaire related to a prescription from an existing patient who has received the prescription, and classifies and evaluates the input responses of the questionnaire according to a type of symptom; and
a prescription error learning unit which learns prescription errors based on medical examination, herbal medicine extraction, prescription evidence mapping, and prescription evidence grade according to the classification and evaluation of the questionnaire responses of the visiting patient response evaluation unit.

8. The medical examination and prescription apparatus for herbal medicine prescription of claim 7, further comprising:
a medical examination correction unit which corrects the existing medical examination based on medical examination error data provided by the prescription error learning unit and provides it to the medical examination unit;
a herbal medicine extraction error correction unit which corrects the herbal medicine extracted in correlation with the medical examination based on herbal medicine extraction error data provided by the prescription error learning unit and provides it to the herbal medicine extraction unit;
a prescription evidence mapping error correction unit which adds new learning prescription evidence to the five types of prescription evidence based on prescription evidence mapping error data provided by the prescription error learning unit and provides it to the prescription evidence mapping unit; and
a prescription evidence grade error correction unit which modifies the grade and rank criteria of the five types of prescription evidence based on prescription evidence grading error data provided by the prescription error learning unit and provides them, or adds a grade and rank criteria of the new learning prescription evidence added by the prescription evidence mapping error correction unit and provides them to the prescription evidence grade determination unit.

9. A medical examination and prescription method for herbal medicine prescription, comprising:
a step for conducting a medical examination in question-and-answer with a patient step by step according to a pre-determined order through a medical examination unit, and receiving an answer to question from the patient according to symptoms of a disease;
a step for sequentially extracting a herbal medicine to be included in a herbal medicine formulation and a herbal medicine to be excluded from the herbal medicine formulation from a herbal medicine database unit in each stage of the medical examination according to the sequential medical examination by using a herbal medicine extraction unit;
a step for sequentially extracting a first prescription candidate group of herbal medicine formulation containing the herbal medicine extracted from the herbal medicine extraction unit from a herbal medicine formulation database unit by using a first prescription candidate group extraction unit;
a step in which a prescription evidence mapping unit sequentially maps five types of prescription evidence to the first prescription candidate group of herbal medicine formulation extracted from the first prescription candidate group extraction unit through the sequential medical examination based on an input of the herbal medicine formulation database unit;
a step in which a second prescription candidate group extraction unit differentiates the first prescription candidate group of herbal medicine formulation mapped by the prescription evidence mapping unit by a grade and a rank according to a degree of mapping of the five types of prescription evidence, and extracts a second prescription candidate group of herbal medicine formulation;
a step in which a prescription providing unit provides an information corresponding to the herbal medicine formulation prescribed by differentially listing according the grade and the rank;
a step in which a visiting patient response evaluation unit receives responses of a questionnaire related to a prescription from an existing patient who has received the prescription, and classifies and evaluates the input responses of the questionnaire according to a type of symptom;
a step in which a prescription error learning unit learns a prescription error, corrects and complements it based on medical examination, herbal medicine extraction, prescription evidence mapping, and prescription evidence grade according to the classification and evaluation of the questionnaire responses of the visiting patient response evaluation unit;
a step in which a medical examination correction unit corrects the existing medical examination based on medical examination error data provided by the prescription error learning unit, and provides it to the medical examination unit;
a step in which a herbal medicine extraction error correction unit corrects the herbal medicine extracted in correlation with the medical examination based on herbal medicine extraction error data provided by the prescription error learning unit, and provides it to the herbal medicine extraction unit;
a step in which a prescription evidence mapping error correction unit adds new learning prescription evidence to the five types of prescription evidence based on prescription evidence mapping error data provided by the prescription error learning unit, and provides it to the prescription evidence mapping unit;
a step in which a prescription evidence grade error correction unit corrects a grade and rank criteria for the five types of prescription evidence based on prescription evidence grading error data provided by the prescription error learning unit and provides them, or adds a grade and rank criteria of the new learning prescription evidence added by the prescription evidence mapping error correction unit, and provides them to a prescription evidence grade determination unit; and
a step for correcting and deriving the first prescription candidate group and the second prescription candidate group of herbal medicine formulation step by step.
